(19) United States
(12) Patent Application Publication
(10) Pub. No.: US 2007/0016032 A1
(43) Pub. Date: Jan. 18, 2007

(54) Microwave devices for treating biological samples and tissue and methods for imaging

(76) Inventor: Gerard Aknine, Coppet (CH)

Correspondence Address: WILEY, REIN & FIELDING, LLP;ATTN: PATENT ADMINISTRATION, 1776 K. STREET N.W., WASHINGTON, DC, 20006, US

(21) Appl. No.: 11/397,706

(22) Filed: Apr. 5, 2006

Related U.S. Application Data
(60) Provisional application No. 60/668,073, filed on Apr. 5, 2005.
(60) Provisional application No. 60/668,059, filed on Apr. 5, 2005.
(60) Provisional application No. 60/676,298, filed on May 2, 2005.

(51) Int. Cl. A61B 8/00

(52) U.S. Cl. 600/437

(57) ABSTRACT

The invention comprises the use of a microwave antenna in conjunction with an ultrasound probe and detector to image below the surface of material, and particularly biological tissue such as skin. A device comprising the microwave antenna and ultrasound probe and detector can be used to image skin tissue and treat skin with microwave energy in order to, for example, reduce the appearance of wrinkles on the surface of the skin.

### RELATED APPLICATION INFORMATION

This application claims priority benefit of U.S. provisional applications 60/668,073 filed on Apr. 5, 2005, 60/668,059 filed Apr. 5, 2005, and 60/676,298 filed May 2, 2005, the entire contents of which are incorporated herein by reference.

### FIELD OF INVENTION

The invention relates to electronic devices and methods for using them to visualize images below the surface of or through a non-transparent medium, or to improve imaging from ultrasound devices. In particular and in one embodiment, a microwave emitting device is operationally coupled to an ultrasound applicator and detector to enhance the ultrasound visulization abilities of tissue, and allow visulization of small tissue characteristics, within the field or focal point area of the emitted microwaves. Previous ultrasound methods and devices rely solely upon the tissue being scanned and its soundwave-refecting properties. The devices and methods of the invention, at least in part, take advantage of an ability to selectively highlight certain areas or tissue using microwaves or electromagnetic fields. The devices and methods provide enhanced ultrasound imaging benefits as well as improved focusing of microwave treatments.

### BACKGROUND OF INVENTION

While the use of ultrasound waves to perform diagnostic imaging of tissue has advanced over the years, the underlying principle remains the same. In general, the systems operate by sending soundwaves through a substance and detecting echoes. In the case of biological tissue, when soundwaves spread through the tissue the borders of differing tissue can be detected if they have different ultrasound attributes. Thus, the borders reflect soundwaves at least partially and to a higher degree if the tissue differences are very great. Differences between soft tissue and bone, for example, are easily detected with ultrasound. The soundwaves or echoes that are reflected back to the ultrasound detector are transformed into electric impulses and then displayed on a monitor.

Certain contrast agents have been used to improve the ultrasound imaging of certain tissues, typically those tissues that are difficult to image or cannot be imaged with conventional ultrasound. The contrast agents take the form of either ingested or injectable compounds or gasses or inserted devices. The utility of these contrast agents is typically limited to specific tissues or areas of the body. Improved devices and methods to visualize or image tissue with ultrasound detection can expand the use of ultrasound in new directions and improve existing diagnostic and treatment possibilities.

### SUMMARY OF THE INVENTION

In one aspect, the invention addresses the need for improved imaging by providing an electronic contrast agent that is capable of working from the surface of the skin or tissue and/or is non-invasive. In a preferred embodiment, a microwave emitting device is combined with an ultrasound device to image the conditions or change in conditions in a tissue or sample, particularly at a desired depth in the sample or tissue. Thus, the methods and devices comprise a non-invasive microwave emitting device and non-invasive imaging device as known in the art or available to one of skill in the art. In certain preferred embodiments, the microwave emitting device can be coupled with or used in conjunction with a specific sonography device and/or system, such as with a 20-MHz B-mode ultrasound scanner or a 5-75 MHz pulser receiver or other system or component (see Hoffmann K., et al., Acta Derm Venereol Suppl (Stockh) 164:3-16 (1991), referring to a DUB 20, Tabema pro Medicum, Lüneburg, Germany; and Jovanovic D L., et al., Arch Dermatol 141: 269-270 (2005) referring to the 20-MHz Dermascan C, Cortex Technology, Hadsund, Denmark; and see Utex Scientific Instruments Inc., Mississauga, Ontario, Canada; and Massa Products Corp., Hingham, Mass.; for exemplary systems and components). The components and elements of the microwave emitting device are selected so that the microwaves can be focused on a particular or selected depth into the tissue or sample, for example, the epidermal layer of the skin and the fibers within the epidermal layer that reflect on the level of tightness of the skin or degree of wrinkles present. Thus, fibers in the epidermal layer can be can be severed or denatured as a result of the microwave treatment and the ultrasound imaging system or device can be used to direct the treatment and/or localize the fibers or cells to be treated.

In one aspect, the invention comprises a method for using a microwave emitting device, where the device comprises a microwave probe or antenna connected to a power supply, and wherein the antenna is optionally formed on or within a substrate. The probe or antenna design takes into consideration the use desired and the permittivity of the components used and the material being treated with microwaves, as well as the power, wavelength and energy desired for that use. In preferred examples, the microwave emitting device is a probe of one or more conduits or wires. Two probes can be used that are separated from each other and that terminate within a substrate having a desired dieletric constant. In other examples, the antenna can be referred to or be a cylindrical antenna, concentric array radial line slot antenna, a slot aperture antenna, an annular slot antenna, a multi-layered concentric aperture antenna, a dual concentric conductor antenna, and an array antennae. In various embodiments, the applicator or antenna can be referred to or be a cylindrical antenna, a concentric array radial line slot antenna, a slot aperture antenna, an annular slot antenna, a patch antenna, a multi-layered concentric aperture antenna, a dual concentric conductor antenna, a coplanar patch antenna, and an array antennae. Examples of these antennae are known to one of skill in the art. A preferred embodiment encompasses a cylindrical antenna or an antennae consisting of an approximately 8-10 um, or 8-10 mm, in diameter concentric array. Examples of these antennae are known to one of skill in the art. In general, the microwave emitting device can be either partially immersed in water or is wetted when in contact with the skin or tissue to be imaged or treated. The surface designed to emit microwaves can also be an aqueous or solution-filled bolus chamber having a surface for contacting a sample or tissue. In this optional embodiment, the bolus chamber is composed of a solid, semi-rigid, or rigid polymer or copolymer, such as polyoxymethylene, designed with a particular dielectric coefficient in mind for the use desired. A method of the invention comprises, in one aspect, positioning the microwave emitting device proximate to or in contact with a sample or tissue under conditions in which the microwaves can penetrate through the bolus and/or penetrate the sample or tissue. Typically, a water, solution, or wetted or moisture layer is applied to the surface contacting the sample or tissue, or the sample or tissue can be treated with water, a solution, or moisture. The layer of material between the surface where microwaves are emitted and the sample or tissue can affect the ability or efficiency of the microwaves to penetrate the sample or tissue, as known in the art. After the microwave emitting device is properly positioned, which may include treating one or more surfaces as just noted or similarly treating those one or more surfaces, one or more microwave pulses are created. The ultrasound imaging device or system can be coupled or can operate in conjunction with the microwave emitting device.

In certain embodiments or any embodiment, the antenna is an annular slot antenna of a size capable of using a power supply to emit approximately twice the frequency desired for use in the sample or tissue, and wherein the dielectric constant of the substrate is approximately 3.5, or between about 2 and 3.5, or between about 2.5 and 3.5. The selection of the antenna, frequency, substrate, and the temperature of the solution in the bolus are such that the microwave emitting device is capable of heating the sample or tissue proximate to the surface for contacting the sample or tissue. Furthermore, the elements of the device can be selected to resonate at the desired operating frequency.

In certain embodiments or any embodiment, the bolus is filled with water, deionized water, distilled water, saline solution, or a solution of silicon in water. Similarly, the substrate for certain or any embodiment is a polymer or copolymer, preferably a POM (polyoxymethylene) polymer having a dielectric coefficient of approximately 3.5. In an embodiment where the solution is deionized or distilled water, the temperature of the water in the bolus can be selected from between about 17° C. and about 28° C. The temperature of the solution or water in the bolus can be regulated by circulating the water or solution into or with a temperature controlled bath external to the bolus and/or by using commercially available temperature controlling devices.

In a preferred embodiment, the method encompasses imaging and/or treating the epidermal layer of skin and/or the antenna is selected to focus the emitted microwaves at a point or area approximately 1.5 mm to 2 mm below the external surface of the derma. The method encompasses a treatment wherein the tissue is skin and the emitted microwaves are directed to an area of the face where aged or wrinkled skin is present, such as around the eyes, lips, chin, neck, and forehead.

In other preferred embodiments, the microwave emitting device and method of ultrasound imaging comprise an ultrasound device to sample the conditions and/or changes in the tissue or sample or structures capable of being detected by ultrasound, particularly at a desired depth in the sample or tissue. Thus, the methods and devices of the invention comprise a noninvasive imaging device as known in the art or available to one of skill in the art. In any embodiment of the invention, the microwave emitting device can be coupled with or used in conjunction with a sonography device or high frequency acoustic wave generator and receiver, such as with a 10-MHz, or 20-MHz, or 25-50 MHz B-mode ultrasound scanner (DUB 20S, tabema pro medicum, Lüneburg, Germany) and/or a pulser receiver (UTEX Scientific Instruments, Mississauga, Ontario, Canada) set to operate a transducer at a desired frequency. Optionally, a water bolus device can be operably connected to the ultrasound transducer probe at the surface that contacts or is proximate to the skin or material to be scanned or imaged. The water bolus need not be operably connected to both the ultrasound transducer and a surface of the microwave emitting device or antenna, but in optional embodiments the water bolus may be used with both the microwave antenna and the ultrasound transducer, for example. In other embodiments, an ultrasound receiver array can be incorporated into the device and system of the invention instead of or in addition to the ultrasound transducer.

In another, general embodiment, the invention encompasses a microwave emitting device for treating skin comprising a microwave antenna capable of emitting a directional, focused beam of radiation, wherein the antenna is embedded in a substrate, wherein the substrate has a back surface and a front surface and the direction of the emitted radiation emanates from the front surface. The device further comprises, in one option, a solution-filled bolus attached to the substrate at the front surface of the substrate, wherein the solution is encased in a solid plastic having a desired dielectric constant and wherein the bolus is about 5 mm in thickness. The antenna is connected to a power source or supply and is capable of sending energy to the antenna resulting in pulses of microwave emissions. The device can further comprise a temperature-controlling device capable of maintaining the bolus at a predetermined temperature. The temperature-controlling device can be operationally coupled to a computer-controlled system for regulating the microwaves emitted in response to the temperature detected in the sample or tissue. In this way, the microwave emitting device can be fine-tuned to emit specific radiation levels to specific depths of a tissue or sample, or to affect tissue at specific depths. In general, the frequency and energy of the microwaves capable of being emitted from the antenna are selected based upon the permittivity of the selected substrate, the solution-filled bolus, the solution in the bolus, and the temperature of the bolus, such that the emitted microwaves penetrate through the bolus and enter skin in contact with the bolus to a desired depth to affect the appearance of the skin, or to penetrate a sample, such as plasma, and heat the sample to eradicate or reduce the level of microorganisms present.

In these and various other uses and embodiments of the invention, the invention includes the steps of using the applicator to generate a focused, converging, and/or quasi-transverse electromagnetic surface wave within the tissue or skin by utilizing the differing dielectric and conductivity characteristics of the skin layers and/or muscle layers and/or fat layers. The amplitude and phase for the individual antenna and/or microwave emitting elements on the device are selected to adjust the focal line within a target region of below the surface of the skin. Simultaneously with the emitting or microwave radiation, the bolus can be used to cool the skin surface to prevent or reduce skin burns and acute pain.

Other embodiments and advantages of the invention are set forth in part in the description that follows, and in part, will be obvious from this description, or may be learned from the practice of the invention.

### BRIEF DESCRIPTION OF THE DRAWINGS

For a more complete understanding of the invention and some advantages thereof, reference is now made to the following descriptions taken in connection with the accompanying drawings in which:

FIG. 1 schematically represents the effect of modulated microwave pulses on ultrasound imaging. In top chart I, the peaks represent ultrasound pulses of, for example, 40-50 Mhz. Chart II represents the echoic results detected by the ultrasound detector.

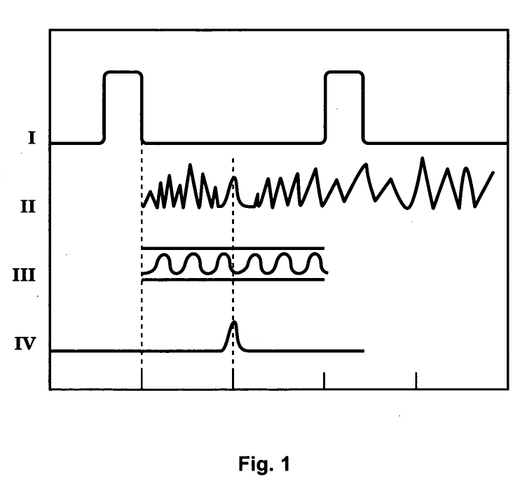

Chart III represents the amplitude modulation microwave energy. Chart IV represents the specific echo at the focal point of the microwave energy that is preferentially amplified and used to image tissue that would otherwise not be amplified.

FIG. 2 depicts the ultrasound image of human skin enhanced with the effects of a microwave emitting device or method of the invention. The areas indicated by (A) and circles are clearly visible fibrous and/or cellular tissue that cannot be imaged by or is substantially invisible with conventional ultrasound techniques. The improved imaging ability of the invention allows treatment to this specific region of the skin, which in turn improves the cosmetic or dermatological effect of the treatment.

FIG. 3 schematically depicts a combination device of the invention, where a microwave source emits microwave energy and an ultrasound source emits ultrasound energy and detects reflected echoes of the ultrasound waves. In this example, a water bolus is used and the microwaves, or the effect of microwaves, are focused at a particular area (FP) below the surface of the skin. A water bolus is not required for the invention, however. It is primarily at the focal point that the microwave energy causes a cross-modulation effect on the tissue that enhances the ultrasound detection and makes the tissue visible for ultrasound imaging. In general, both the positive and negative reflected ultrasound waves are used to image the focal point area, whereas conventional ultrasound detection typically uses only the positive waves. One of skill in the art is familiar with various ultrasound pulser devices, receivers, and imaging systems and software, including those using the A-mode and B-mode ultrasound and the use of reflected ultrasound waves to construct images.

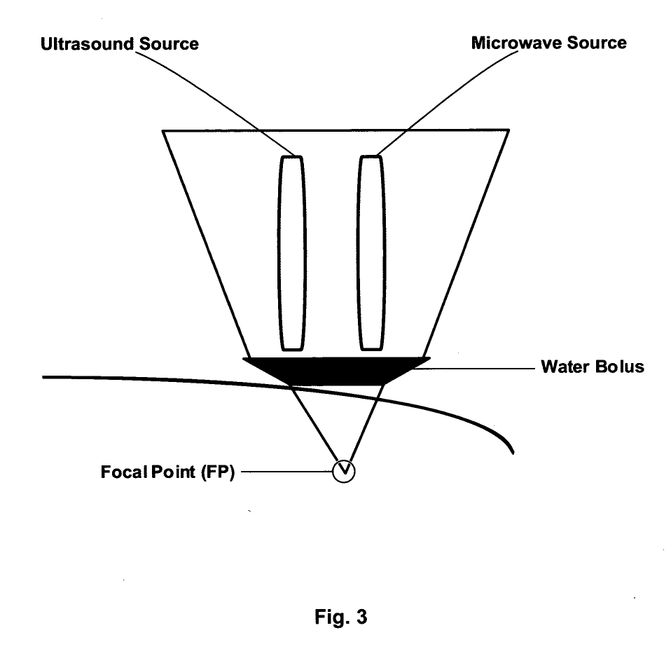

FIG. 4 schematically depicts the device of FIG. 3 connected to an ultrasound imaging system and placed over the surface of a medium, such as skin. The areas numbered 1 and 2 on the handheld probe refer to the microwave emitting device (1) and the ultrasound transducer/receiver (2). The microwave energy is shown focusing to a point below the surface of the medium. The part-circle waves represent the ultrasound waves being sent into the medium from the transducer and being reflected back from the area where the microwave energy is focused. In this general way, methods and devices of the invention can be used to provide improved imaging or detection of features or characteristics below the surface of a medium. In particular, the devices and methods are used with living or animal tissue, however, other medium can also be used, such as composite material, metals and alloys, where imaging can detect material flaws, cracks, or material weaknesses.

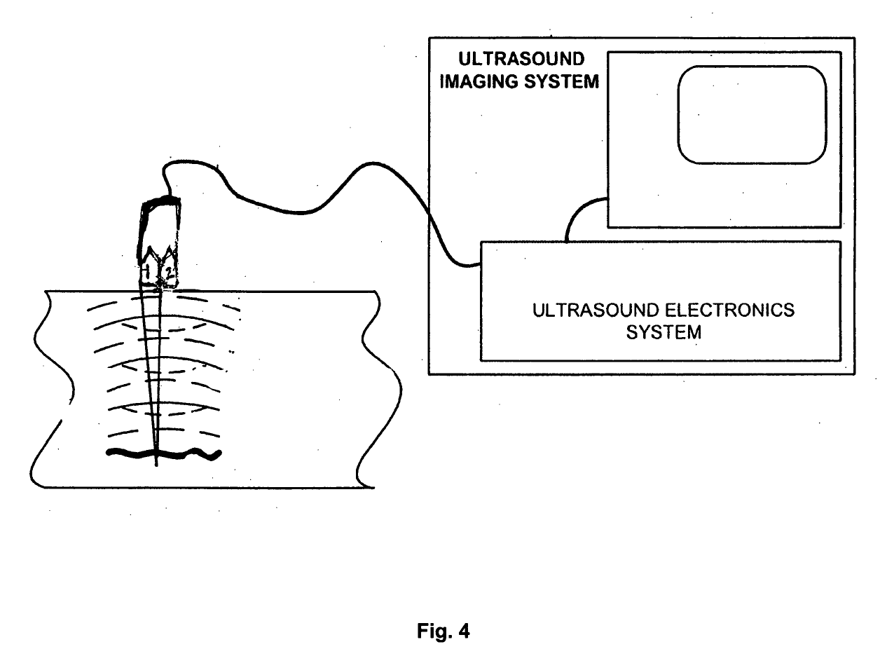

FIG. 5 is a diagram of an exemplary imaging device of the invention. The ultrasound transducer is typically connected to a microwave emitting device or antenna and moved over the surface or along a plane together to scan a surface (motion control). Here, the movement or displacement of the transducer and antenna unit, for example, is about 12.8 mm. In one example, 384 points are sampled or received over each 12.8 mm pass, and a comparison of the feedback ultrasound or echoes made between those when microwave energy is emitted versus when it is not emitted. The difference can be resolved with algorithms or software methods through a processor, for example, and displayed as an ultrasound image showing the areas where microwave modulation occurs through the focused microwave energy within the medium. The components of the pulser are standard and can be selected from those available in the art or known to one of skill in the art.

FIG. 6 depicts a directional applicator or antenna for emitting energy in only one direction or from only one plane. In A, (31) represents the surface where the emitted energy is directed from and (30) depicts a blocking or shielding surface. A cable (32) connects to a probe within the interior of the cylindrical apparatus, where the probe is positioned at a desired position or distance (33) away from the blocking or shielding surface (30). The view in B is a cross-section of the same apparatus. The cable (32) connects to probe (35). Here, an optional cylindrical gap region is used and formed around an inner area, where gaps (37) are shown formed from walls (38). A cylindrical antenna without a gap region can also be used. In either case, the inner area is filled with appropriate dielectric material or resin (36) to allow the energy to be transmitted. The size of the inner area (36) can be selected for a particular probe or frequency or power source. The distance (39) and (40) can be selected for the particular probe, geometry and size of the gaps and inner area, and the thickness and material in the inner area and other components of the apparatus. Surface (34) prevents the propagation of waves, so that energy is emitting in only one direction, or at least substantially only one direction. This provides advantageous properties for a device that used by a person desiring energy to be emitted in a specific area or in only one direction. The distance (41) and (42) can be also varied to control the energy emitted and a focal point of emitted energy.

FIG. 7 is a diagram of the microwave emitter device or antenna and its electronic and wave control components. The elements can be selected from those available in the art or known to one of skill in the art. The focused microwaves or energy or “Beam” is depicted below the waveguide antenna, which can be one as shown in FIG. 6.

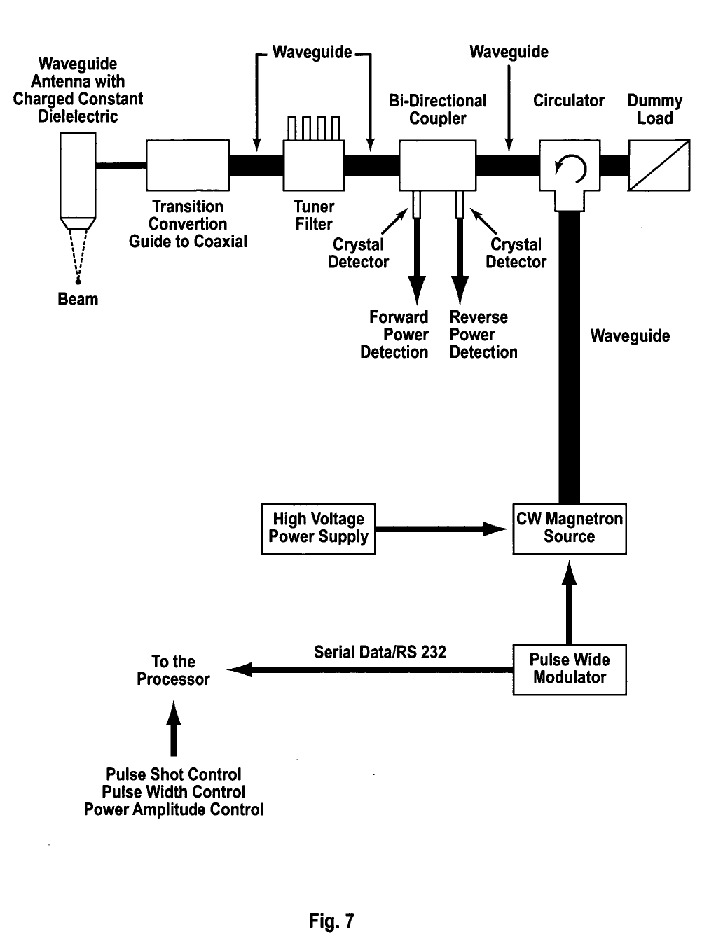

FIG. 8 is an exemplary hand-held unit showing an internal device for moving the probe or antenna back and forth over the surface of a medium, such as skin or tissue. Rotating groove (56) forces the entire internal unit (57) to move back and forth. As stated, a preferred example moves 12.8 mm each pass, but other lengths can be selected based upon the medium or desired results. Surface (58) is where energy is emitted and is often in contact with the surface of the medium, preferably in contact with water or other solution to improve transmission.

FIG. 9 shows an underside view of the hand-held device of FIG. 8, where two separate elements are included. One of the elements (60) can be a probe or antenna, such as a cylindrical antenna or as described in FIG. 6, and the other an ultrasound transducer (59) for imaging the skin or tissue or material. The microwave emission in combination with the ultrasound imaging can be used together to both visualize tissue and specific features of skin and tissue that are not easily detectable with ultrasound alone, and to pin-point the placement of the emitted microwave energy under the surface of the skin or tissue.

FIG. 10 shows a similar view to that of FIG. 9, where the ring of the cylindrical directional microwave antenna (60) can be seen, such as one as exemplified in FIG. 6. Ultrasound transducer (59) and arm or rod (61) for moving both transducer (59) and antenna (60) together over a surface are shown. Surface (62) can be covered or wetted, with water, aqueous solution or gels, to enhance transmission of ultrasound and microwaves.

### DETAILED DESCRIPTION OF THE INVENTION

Throughout this disclosure, documents and references to information are cited and the information contained in the cited information can be relied upon by one of skill in the art to make and use aspects of this invention. To the extent allowed and when necessary, parts of or the entire contents of the information can be incorporated into this disclosure. However, nothing in the cited information should alter a specific definition of the invention stated here. Specifically incorporated by reference is their entirety are the disclosures of U.S. provisional applications 60/668,073 filed on Apr. 5, 2005, 60/668,059, filed Apr. 5, 2005, and 60/676,298 filed May 2, 2005.

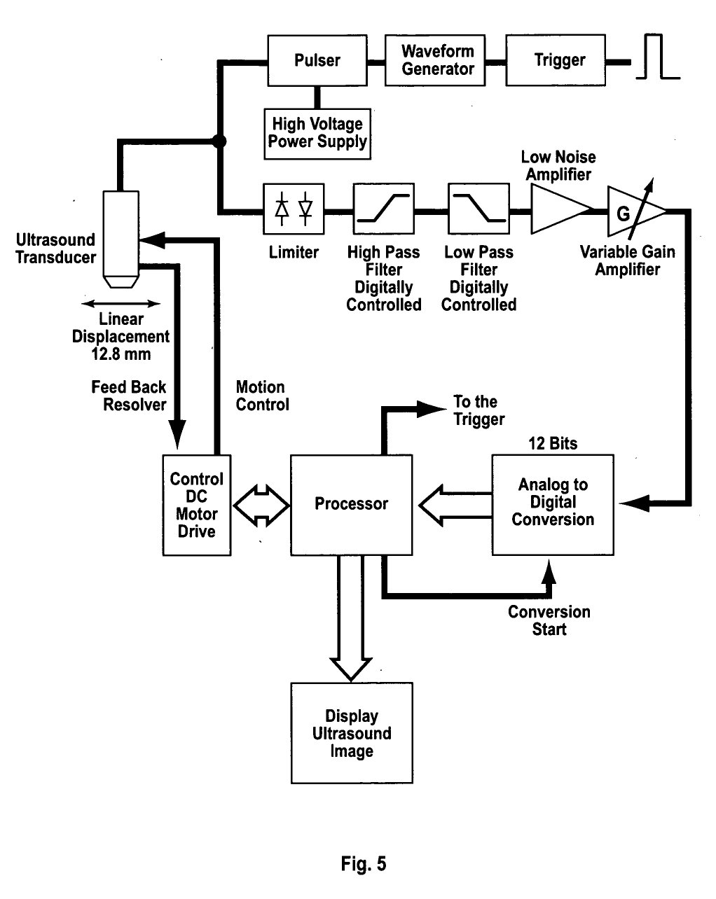

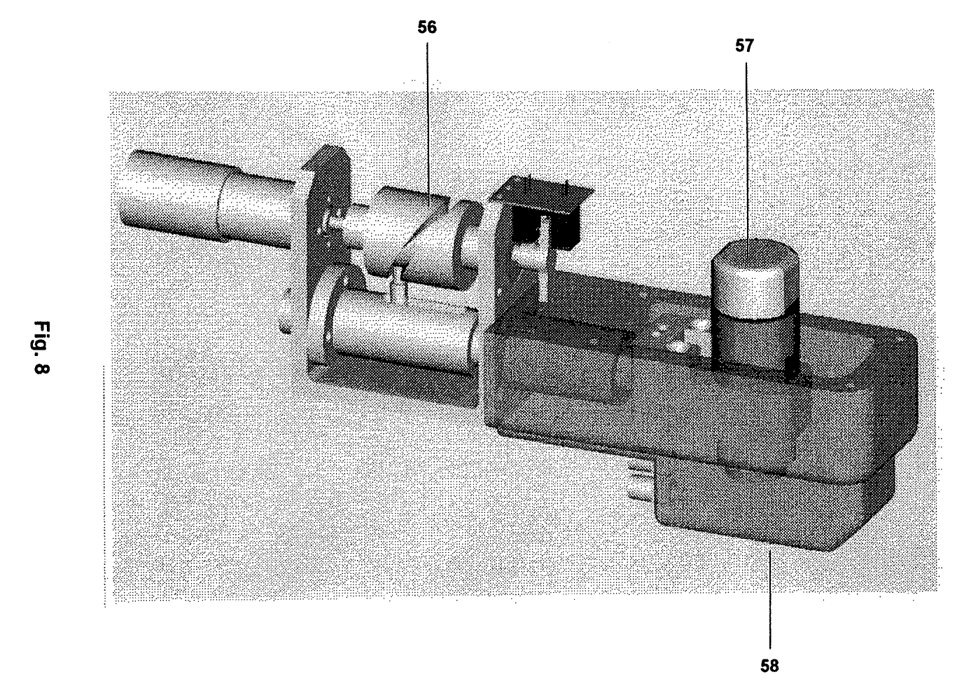

The devices and methods of the invention can be adapted and used with a variety of medium, tissues and samples and at a variety of penetrating depths. While skin tissue is discussed in general here, other tissue can be equally used. In addition, samples of other mediums or compositions that can be sampled through ultrasound or low frequency scanning can be selected and used. Thus, while skin tissue is discussed in particular, the invention is not limited to use with any particular medium, tissue or sample. In that regard, the method of enhancing the ultrasound detection of a sample can comprise selecting the frequency of electromagnetic energy to focus at certain depth in the sample preferably, but not limited to, microwave energy. The amplitude of applied, focused energy is modulated and directed to an area of the sample. An ultrasound scanner is used to send ultrasound waves and detect ultrasound reflections. A computer-controlled or other ultrasound receiver and processing system is used to determine the amplitude of focused energy that improves the detection of structures within the area of the sample compared to the detection without applying the focused energy. Typically, a comparison between ultrasound feedback with and without microwave energy emission leads to enhanced imaging, as the difference between the two can be compared and identified. Thus, as shown in FIGS. 5 and 8, for example, the microwave waveguide antenna and transducer together move back and forth over a surface, and one pass of 12.8 mm without microwave emission is compared to one pass of 12.8 mm with microwave emission. One of skill in the art is familiar with using or adapting software to compare images to subtract or identify difference between samples, and such difference images can be displayed on a monitor. In this general method, the invention can be used on a number of different samples and not solely on biological samples or tissue samples.

However, in preferred examples the method encompasses the use of microwave energy and skin, especially skin where treatment or wrinkle-reducing methods are desired. A microwave pulse can be about 1 msec in length or other duration, for example 20 ns to 2 secs, or 20 ns to 30 ms, or 30 ms to 500 ms, or 500 ms to 1 sec. In other embodiments, the pulses can be longer. The microwave pulse can be of a frequency and energy that penetrates through the skin and is capable of penetrating to a desired depth of the skin. For example, the frequency can be 200 MHz to 2.5 GHz, or 300 MHz to 1.3 GHz, or 400 MHz to 950 MHz, or 400 MHz to 500 MHz, or 850 MHz to 950 MHz. Preferred frequencies are 433-434 MHz and about 915 MHz. Furthermore, in some optional embodiments, the antenna and generator can be calibrated, tuned, and/or matched at a frequency X but operated at a frequency different from X, for example half X or twice X, or within a range from about half X to about twice X. Typically, the frequency selected for the generator and waveguide for a λ/2 dipole type antenna is twice the frequency desired to be penetrating through the sample or tissue. Thus, in one embodiment, the frequency selected is about 866 MHz for a dual concentric conductor antenna, however one of skill in the art is capable of selecting, testing, and using many other possible frequencies to affect the temperature of tissue (see, for example, Mizushina et al., “Effects of water-filled bolus on the precision of microwave radiometric measurements of temperature in biological structures,” Microwave Symposium Digest, 1900, IEEE MTT International; incorporated herein by reference). In a generic version of the microwave emitting device, the Power supplied from the microwave power source can applied in a range of 0.1-10,000 Watts per aperture and preferably, in a range of 5-50 Watts per aperture, and less than 40 Watts per aperture. The power is applied either in short high power pulses or preferably, at a continuous wave frequency in the range of 300-5000 MHz or more, in a range of 430-5000 MHz, and most preferably, at a continuous wave frequency of about 433 MHz or about 866 MHz. Treatment is continued for a desired amount of time in accordance with the desired results, preferably, less than 1 second to 5 minutes for high power pulses, and more preferably, for durations of 30 minutes to 4 hours for moderate power, or for extended periods (e.g. overnight) at even lower average power levels.

The following Examples, and forgoing description, are intended to show merely optional configurations for the devices of the invention. Variations, modifications, and additional attachments can be made by one of skill in the art. Thus, the scope of the invention is not limited to any specific Example or any specific embodiment described herein. Furthermore, the claims are not limited to any particular embodiment shown or described here.

### EXAMPLE

### Dermatology Imaging

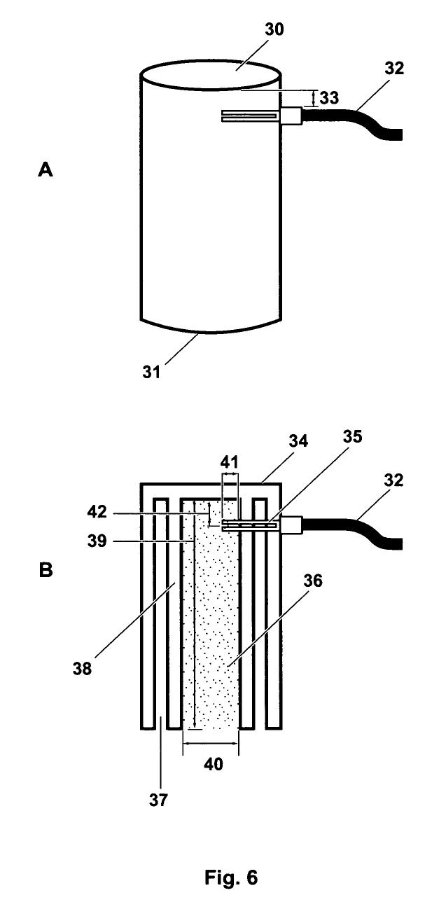

In one embodiment designed for cosmetic treatments of skin or other tissue treatments, an uni-directional antenna, as in a cylindrical antenna or the antenna of FIG. 6, is used.

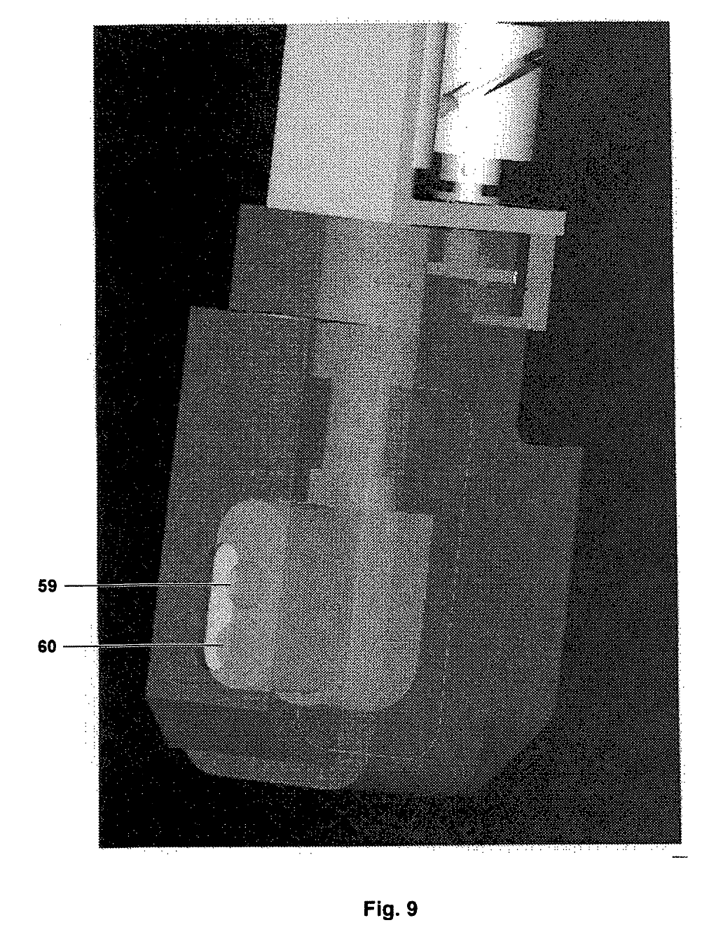

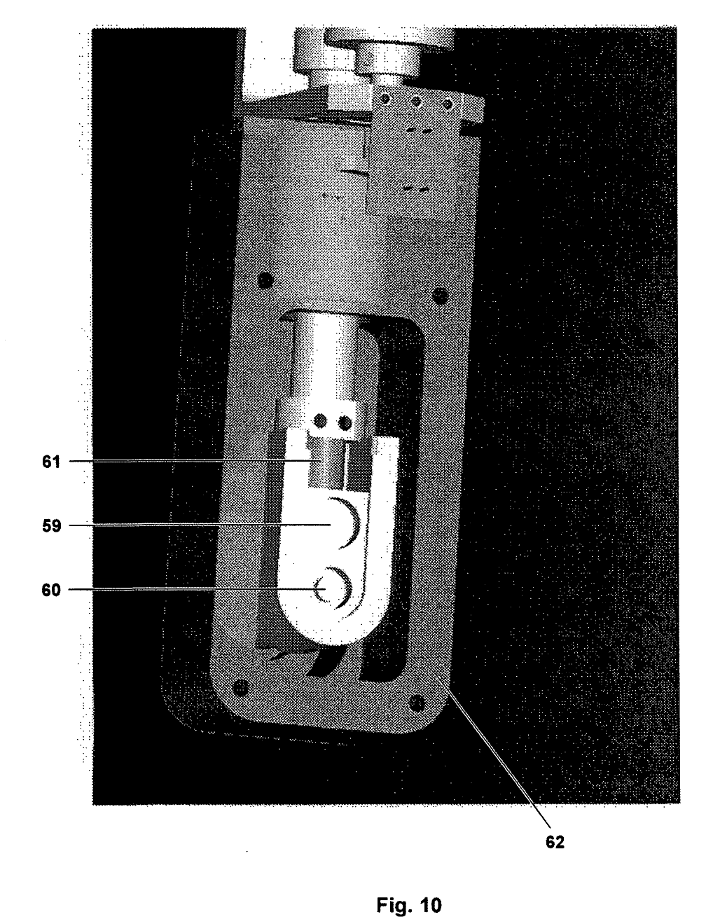

This antenna employs a microwave short circuit surface or side to prevent the emission of energy on one side. Thus, the energy is solely emitted through the open, field-generating end through the dielectric resin of the interior chamber. Typically, the coaxial cable, again 50 ohm, is connected to a gold probe of a particular length and configuration and placed a particular distance from the end of the circular waveguide device, configured for 5.8 GHz in this example, although other frequencies can be selected. An e-field can be generated that penetrates skin tissue or a desired material or composite. A cylindrical antenna, and the particular device of FIG. 6, can be specifically useful when the field-generating end is either submerged in water or aqueous solution or sufficiently wetted, as by a gel, to make contact with the surface of the skin or tissue to be treated. The antenna device of FIG. 6 can be incorporated into a hand-held apparatus as shown in FIGS. 8, 9, and 10. In a preferred embodiment, the antenna device is coupled with an ultrasound probe to image the area being treated. In one example, this device is designed to be used for the treatment of skin and particularly the reduction or elimination of wrinkles, fissures, or fine lines in the face of a patient. The treatment regimen consists of microsecond pulses at about 866 MHz designed to penetrate to a depth of about 1.5 to about 2.0 mm below the surface of the skin.

One of skill in the art can select any appropriate or desired ultrasound scanning equipment and imaging system (see Hoffmann K., et al., Acta Derm Venereol Suppl (Stockh). 164:3-16 (1991), referring to a DUB 20; Taberna pro Medicum, Lüneburg, Germany; and Jovanovic D L., et al., Arch Dermatol 141: 269-270 (2005) referring to the 20-MHz Dermascan C, Cortex Technology, Hadsund, Denmark).

As shown in the FIGS. 8-10, a high-resolution ultrasound scanner (transducer/detector) can be mounted on a moving element together with the microwave probe or applicator. The two elements move together to emit microwaves and ultrasound waves over an area approximately 10 mm wide, and the reflected ultrasound waves detected directed to an image processing center or computer. The proximity of the microwave emitting device to the ultrasound scanning probe can be varied, and in the examples of FIGS. 8 to 10 is approximately 10 to about 30 mm apart center to center, or 10 to 25 mm apart, or 15 to 20 mm apart. Furthermore, the distance traveled or moved or displaced in an embodiment where the handheld unit comprising the ultrasound transducer and microwave emitting device move together can be form about 5 mm to about 50 mm, or about 10 mm to about 30 mm, or about 10 mm to about 20 mm. The handheld unit containing the optionally moving ultrasound transducer and microwave emitter are connected to a controller and image processing center, which sends images to a computer or PC for display on a monitor.

The type of ultrasound transducer selected can also be modified depending on the type of tissue and depth of treatment penetration desired. As one of skill in the art knows, certain ultrasound frequencies are more useful for certain types of tissue and for penetrating or detecting signal or echoes from certain depths into a tissue. For example, in examples where breast tissue is imaged, a depth of about 10 to 12 cm is desired and a frequency of about 5 MHz may be used. In other tissue, one of a range of frequencies can be selected, for example any frequency between about 5 MHz and 75 MHz, or 5-50 MHz, or 10-40 MHz, or 30-40 MHz, or 20-50 MHz. In addition, the configuration and material used for the ultrasound transducer can also effect its use or optimal signal detection from different tissue (see, in general, Wells, IEEE Engineering in Medicine and Biology, September/October 2000, pp. 14-20). Digital image processing is also known in the art and can be used through the process to improve image quality and image processing effects. Furthermore, the waveform used can be the so-called chirp waveform or coded waveform to improve imaging, as known in the art. The chirp waveform can even be used on a percentage of pulses as desired to improve imaging.

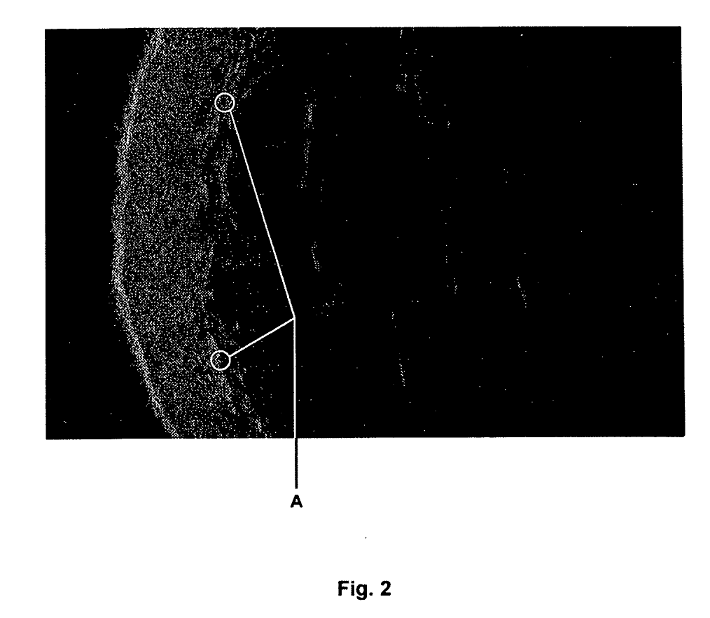

The image shown in FIG. 2 depicts tissue below the surface of skin from a computer monitor operating a high resolution ultrasound apparatus (for example, a 10-MHz or 20-MHz B-mode ultrasound scanner DUB 20S, taberna pro medicum, Lüneburg, Germany) combined with the microwave emitter of the invention. A hand held, combined ultrasound scanner and microwave applicator as mentioned above is used over the arm surface. Ultrasound detection is processed through an image processing program comparing the ± microwave emitted ultrasound samples. The differences between ± microwave emission show the tissue at the focal point area with the most resolution and can be used to identify structural or biochemical features or fibers not typically visible through conventional ultrasound.

The arrows in FIG. 2 point to regions of the tissue that cannot be adequately imaged with conventional ultrasound. Thus, the invention provides a fine resolution image of tissue that can be treated with noninvasive or cosmetic dermatological methods, such as Rf or microwave treatment or focused Rf or microwave treatments. As used herein, Rf or microwave can mean a desired frequency or range between 200 MHz and 1.2 GHz or compatible with the uses disclosed herein.

One skilled in the art can devise and create numerous other examples according to this invention. Examples may also incorporate additional imaging, thermometry, and other elements known in the art. One skilled in the art is familiar with techniques and devices for incorporating the invention into a variety of devices and of designing improved devices though the use of the concepts presented here.

What is claimed is:

1. A method for ultrasound imaging comprising
emitting microwaves from an antenna or applicator at the surface of a sample or tissue under conditions in which the microwaves can penetrate through the sample or tissue and where microwave pulses of about 1 msec in length and of a desired frequency and energy to penetrate a desired depth into the sample or tissue;
emitting ultrasound pulses to the same sample or tissue from an ultrasound scanner; and
detecting the reflected ultrasound waves or feedback,
whereby at least part of the tissue or sample subjected to microwave energy is more visible in an image constructed from the reflected ultrasound waves than it is in tissue or sample where no microwave energy is emitted.

2. The method of claim 1, wherein the tissue is skin.

3. The method of claim 1, wherein the ultrasound scanner utilizes a frequency between about 5 MHz and about 75 MHz.

4. The method of claim 1, wherein the antenna or applicator is a directional antenna for emitting energy in substantially one direction or from only one plane, wherein the antenna or applicator design comprises at least one cylinder formed around at least probe connected to a waveguide, and the cylinder contains a substrate having a desired dielectric constant.

5. The method of claim 4, wherein the substrate selected is a polymer or copolymer.

6. The method of claim 5, wherein the substrate is a POM (polyoxymethylene) polymer.

7. The method of claim 4, wherein the antenna or applicator is selected to emit microwaves that penetrate to an area approximately 1 mm to 20 mm below the external surface of skin.

8. The method of claim 7, wherein the area is between about 1 mm to about 2 mm below the external surface of skin.

9. A method of enhancing the ultrasound detection of tissue comprising
selecting a frequency of electromagnetic energy to penetrate to a certain depth in a tissue or sample;
modulating the amplitude of the electromagnetic energy;
detecting ultrasound reflections from an ultrasound scanner;
determining the amplitude of focused energy that improves the detection of structures within the area of tissue compared to the detection without applying the focused energy.

10. The method of claim 9, wherein microwave energy is used.

11. The method of claim 9, wherein the ultrasound scanner employs a frequency between about 5 and 100 GHz.

12. The method of any one of claims 9 to 11, wherein the tissue is skin.

13. A device for ultrasound imaging comprising
a microwave applicator or antenna capable of emitting energy, wherein the applicator or antenna comprises a selected dielectric constant and the direction of the emitted radiation is from a front surface of the applicator or antenna;
optionally a solution-filled bolus attached to the substrate at the front surface, wherein the solution is encased in a solid plastic having a desired dielectric constant and wherein the bolus is about 5 mm in thickness;
a power supply connected to the applicator or antenna and capable of sending energy to the applicator or antenna resulting in pulses of microwave emissions;
an ultrasound scanner comprising a transducer and a detector; and
an image processor capable of producing an image from the detected ultrasound waves,
wherein the frequency and amplitude of the focused microwave energy is capable of emitting energy to a desired tissue below the surface of skin, and wherein the desired tissue is visible in the ultrasound image more than or substantially more than when no focused microwave energy is emitted to the tissue.

14. The device of claim 13, wherein the frequency and energy of the microwaves capable of being emitted from the applicator or antenna are selected based upon the permittivity of the selected substrate, optionally the solution-filled bolus, the solution in the bolus, and the temperature of the bolus, whereby the emitted microwaves penetrate the skin to a desired depth.

15. The device of claim 13, wherein the applicator or antenna is a cylindrical antenna.

16. The device of claim 13, wherein the applicator or antenna and ultrasound scanner are connected to a moving arm.

17. The device of claim 16, wherein the moving arm is capable of scanning over a selected area or a selected distance.

18. The device of claim 16, wherein the applicator or antenna and ultrasound scanner are at a fixed distance to each other.

19. The device of claim 18, wherein the moving arm is capable of scanning over a desired area.

20. A method of image processing using a high resolution ultrasound transducer/receiver combined with a microwave emitting device, comprising
emitting microwave energy at particular time points;
emitting ultrasound pulses;
detecting reflected ultrasound waves at time points when microwaves are emitted and when microwaves are not emitted;
comparing the + microwave detected feedback and the − microwave detected feedback to construct a difference image; and
displaying the difference image on a monitor.

21. The method of claim 20, wherein the ultrasound pulses are at a frequency between about 5 MHz and about 75 MHz.

22. The method of claim 20, wherein the frequency of microwave energy is between about 300 MHz and 5 GHz.

23. The method of claim 20, wherein the frequency of microwave energy is between about 300 MHz and 2.54 GHz.

24. The method of claim 20, wherein the frequency of microwave energy is about 2.54 GHz.

25. The method of claim 20, wherein the frequency of microwave energy is about 434 MHz.

26. The method of claim 20, wherein the frequency of microwave energy is about 915 MHz.
